(19) United States
(12) Patent Application Publication
(10) Pub. No.: US 2010/0247622 A1
(43) Pub. Date: Sep. 30, 2010

(54) TREATMENT REGIME FOR PROLIFERATIVE DISORDERS

(75) Inventors: Matthew C. Coffey, Calgary (CA); Bradley G. Thompson, Calgary (CA)

Correspondence Address: McKeon Meunier Carlin & Curfman LLC, 817 W. Peachtree Street, Suite 900, Atlanta, GA, 30308, US

(73) Assignee: ONCOLYTICS BIOTECH INC., Calgary, AB (CA)

(21) Appl. No.: 12/678,851

(22) PCT Filed: Oct. 22, 2008
(86) PCT No.: PCT/CA2008/001865

§ 371 Date: Mar. 18, 2010

Related U.S. Application Data
(60) Provisional application No. 60/981,716, filed on Oct. 22, 2007.

(51) Int. Cl. A61K 39/395; A61K 35/76; A61K 9/127; A61P 35/00

(52) U.S. Cl. 424/450; 424/93.6; 424/93.2; 424/93.3; 424/173.1; 424/158.1

(57) ABSTRACT

Provided herein are methods for treating or ameliorating a proliferative disorder in a subject. The method contains the steps of administering to the subject one dose of an immunosuppressive agent followed by administering to the subject one to five doses of a reovirus. The immunosuppressive agent is administered to the subject at least about 72 hours prior to administration of the reovirus. Also provided herein are methods for treating or ameliorating a proliferative disorder in a subject including the steps of administering to the subject an oncolytic virus and a B-cell modulating agent. Also provided are kits and pharmaceutical compositions containing an oncolytic virus and at least one B-cell modulating agent.

### CROSS-REFERENCE TO RELATED APPLICATIONS

This application claims priority to U.S. Provisional Application No. 60/981,716, filed Oct. 22, 2007, which is incorporated by reference herein in its entirety.

### BACKGROUND

In spite of numerous advances in medical research, cancer remains a leading cause of death. In the search for treatment strategies for cancer, oncolytic virus therapy has recently emerged as a viable approach to specifically kill tumor cells. Unlike conventional gene therapy, it uses replication competent viruses that are able to spread through tumor tissue by virtue of viral replication and concomitant cell lysis, providing an alternative treatment for cancer. Viruses that selectively replicate and kill cancer cells have now been identified.

Oncolytic viruses may utilize multiple mechanisms of action to kill cancer cells—cell lysis, cell apoptosis, anti-angiogenesis and cell necrosis. The virus infects the tumor cell and then begins to replicate. The virus continues to replicate until the host cell's membrane lysis (bursts) as the tumor cell can no longer contain the virus. The tumor cell is destroyed and the newly created viruses are spread to neighboring cancer cells to continue the cycle. Oncolytic viruses are intended to replicate only in cancer cells and to pass through normal tissue without causing harm. Hence, once all the tumor cells are eradicated, the oncolytic virus no longer has the ability to replicate and it is cleared from the body.

Over the past few years, new insights into the molecular mechanisms of viral cytotoxicity have provided the scientific rationale to design more effective oncolytic viruses. Recent advances in molecular biology have allowed the design of several genetically modified viruses, such as adenovirus and herpes simplex virus that specifically replicate in, and kill, tumor cells. Viruses with intrinsic oncolytic capacity are also being evaluated for clinical purposes.

### SUMMARY

Provided herein are methods for treating or ameliorating a proliferative disorder in a subject. The method includes the steps of administering to the subject one dose of an immunosuppressive agent followed by administering to the subject one to five doses of a reovirus. The immunosuppressive agent is, optionally, administered to the subject at least about 72 hours prior to administration of the reovirus. The treatment regime is designed to optimize therapeutic effectiveness of the reovirus.

Also provided herein are methods for treating or ameliorating a proliferative disorder in a subject including the steps of administering to the subject an oncolytic virus and a B-cell modulating agent. Also provided are kits and pharmaceutical compositions containing an oncolytic virus and at least one B-cell modulating agent.

### DETAILED DESCRIPTION

Reovirus refers to any virus classified in the reovirus genus, whether naturally occurring, non-naturally occurring, modified, reassortant or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kilo base pairs. The individual RNA segments vary in size. Three distinct but related types of reoviruses have been recovered from many species. All three types share a common complement-fixing antigen.

The human reovirus consists of three serotypes: type 1 (strain Lang or T1L), type 2 (strain Jones, T2J), and type 3 (strain Dearing or strain Abney, T3D). The three serotypes are easily identifiable on the basis of neutralization and hemagglutinin-inhibition assays.

The reovirus is naturally occurring or non-naturally occurring. A reovirus is naturally-occurring when it can be isolated from a source in nature and has not been intentionally modified by humans in the laboratory. For example, the reovirus optionally is from a field source, that is, from a human who has been infected with the reovirus. A reovirus is non-naturally occurring when it has been modified or manipulated in a laboratory. For example, a non-naturally occurring reovirus is selected from a laboratory strain or is mutagenized. The non-naturally occurring reovirus is optionally mutagenized to enhance oncolytic activity. As used herein, the phrase enhanced oncolytic activity refers to a reovirus that is more active than a wild-type or control reovirus. As used herein, oncolytic activity refers to the ability of the reovirus to kill neoplastic cells. Thus, for example a reovirus with enhanced oncolytic activity kills at least about 1.5, 2, 5, 10, 20, 50, 75, 100 times or any amount in between 1.5 to 100 times more neoplastic cells as compared to a control or wild-type reovirus. Optionally, the reovirus with enhanced oncolytic activity kills at least about 100 times or more neoplastic cells as compared to a control or wild-type reovirus.

The reovirus is optionally modified but still capable of lytically infecting a cell, e.g., a mammalian cell, having an active ras pathway. For example, the reovirus is chemically or biochemically pretreated (e.g., by treatment with a protease, such as chymotrypsin or trypsin) prior to administration to the proliferating cells. Pretreatment with a protease removes the outer coat or capsid of the virus and optionally increases the infectivity of the virus. The reovirus is optionally coated in a liposome or micelle. For example, the virion is treated with chymotrypsin in the presence of micelle-forming concentrations of alkyl sulfate detergents to generate a new infectious subvirion particle (ISVP).

The reovirus is optionally a recombinant or reassortant reovirus resulting from the recombination/reassortment of genomic segments from two or more genetically distinct reoviruses. Recombination/reassortment of reovirus genomic segments may occur in nature or in a laboratory following infection of a host organism with at least two genetically distinct reoviruses. Recombinant reoviruses are optionally generated in cell culture, for example, by co-infection of permissive host cells with genetically distinct reoviruses.

Accordingly, provided herein are recombinant or reassortant reoviruses containing genome segments from two or more genetically distinct reoviruses, including but not limited to, human reoviruses, such as type 1 (e.g., strain Lang), type 2 (e.g., strain Jones), and type 3 (e.g., strain Dearing or strain Abney), non-human mammalian reoviruses or avian reoviruses. Also provided are recombinant reoviruses containing genome segments from two or more genetically distinct reoviruses wherein at least one parental virus is genetically engineered, comprises one or more chemically synthesized genomic segment, or has been treated with chemical or physical mutagens.

Recombinant reoviruses that have undergone recombination in the presence of chemical mutagens, including but not limited to, dimethyl sulfate and ethidium bromide, or physical mutagens, including but not limited to, ultraviolet light and other forms of radiation are provided. Recombinant reoviruses that comprise deletions or duplications in one or more genome segments, that comprise additional genetic information as a result of recombination with a host cell genome, or that comprise synthetic genes are also provided.

The reovirus is optionally modified by incorporation of mutated coat proteins or polypeptides, such as, for example, into the virion outer capsid. The proteins are mutated by replacement, insertion, or deletion. Replacement includes the insertion of different amino acids in place of the native amino acids. Insertions include the insertion of additional amino acid residues into the protein or polypeptide at one or more locations. Deletions include the deletion of one or more amino acid residues in the protein or polypeptide. Such mutations may be generated by methods known in the art. For example, oligonucleotide-site-directed mutagenesis of the gene encoding for one of the coat proteins results in the generation of the desired mutant coat protein. Expression of the mutated protein in reovirus-infected-mammalian cells in vitro, such as COS 1 cells, results in the incorporation of the mutated protein into the reovirus virion particle.

Optionally, the provided methods include the use of reoviruses with mutations (including insertions, substitutions, deletions or duplications) in one or more genome segments. Such mutations can comprise additional genetic information as a result of recombination with a host cell genome or comprise synthetic genes. For example, mutant reoviruses as described herein can contain a mutation that reduces or essentially eliminates expression of a lambda3 polypeptide or that results in the absence of a functional lambda3 polypeptide as described in U.S. Ser. No. 12/124,522, which is incorporated by reference herein in its entirety. A mutation that eliminates expression of a lambda3 polypeptide or that results in the absence of a functional lambda3 polypeptide can be in the nucleic acid encoding the lambda3 polypeptide (i.e., the L1 gene) or in a nucleic acid that encodes a polypeptide that regulates the expression or function of the lambda3 polypeptide. Optionally, the reovirus as disclosed herein is identified as IDAC Accession No. 190907-01.

The reovirus is optionally a reovirus modified to reduce or eliminate an immune reaction to the reovirus. Such a modified reovirus is termed immunoprotected reovirus. Such modifications include packaging of the reovirus in a liposome, a micelle, or other vehicle to mask the reovirus from the immune system. Optionally, the outer capsid of the reovirus virion particle is removed since the proteins or polypeptides present in the outer capsid are the major determinant of the host humoral and cellular responses.

Reoviruses can be purified using standard methodology. See, for example, Schiff et al., “Orthoreoviruses and Their Replication,” Ch 52, in Fields Virology, Knipe and Howley, eds., 2006, Lippincott Williams and Wilkins; Smith et al., 1969, Virology 39(4):791-810; and U.S. Pat. Nos. 7,186,542; 7,049,127; 6,808,916; and 6,528,305. As used herein, purified mutant reoviruses refer to reoviruses that have been separated from cellular components that naturally accompany them. Typically, reoviruses are considered purified when they are at least 70% (e.g., at least 75%, 80%, 85%, 90%, 95%, or 99%) by dry weight, free from the proteins and other cellular components with which they are naturally associated.

Administration of a virus to a subject refers to the act of administering the virus to a subject in a manner so that it contacts the target neoplastic cells. The route by which the virus is administered, as well as the formulation, carrier or vehicle, depend on the location as well as the type of the target cells. A wide variety of administration routes are employed and are discussed below in further detail. Infection by virus refers to the entry and replication of virus in a cell. Similarly, infection of a tumor by a virus refers to the entry and replication of virus in the cells of the tumor.

Reovirus is used effectively as an oncolytic agent in a number of animal models. Severe combined immune deficient mice (SCID) mice bearing tumors established from v-erbB-transformed murine NIH 3T3 cells or human U87 glioblastomas cells have been treated with reovirus (Coffey, M C et al 1998 Science 282: 1332). A single intratumoral injection of virus resulted in regression of tumor. Animals given bilateral U87 tumor xenografts were given a single unilateral injection of reovirus into the ipsilateral tumor that resulted in reduction in the contralateral tumor. This reduction in the remote tumor site is the result of systemic spread of the virus. Treatment of immune-competent C3H mice bearing tumors established from ras-transformed C3H-10T1/2 cells also resulted in tumor regression upon a series of viral injections (Coffey, M C et al 1998 Science 282: 1332).

Provided herein are methods for treating or ameliorating a proliferative disorder in a subject. Administration of an immunosuppressive agent to a subject followed by administration of the reovirus allows reduction in the concentration and/or frequency of reovirus treatments. The method contains the steps of administering to the subject one dose of an immunosuppressive agent followed by administering to the subject one to five doses of a reovirus. Thus, one dose, two doses, three doses, four doses or five doses of reovirus are administered. The immunosuppressive agent is administered at least 72 hours prior to administration of the reovirus. Optionally, the immunosuppressive agent is administered from about 96 to 72 hours or at any time point in between 96 and 72 hours prior to administration of the reovirus. Optionally, the immunosuppressive agent is administered at 72 or 96 hours prior to administration of the reovirus. Alternatively, the immunosuppressive agent is administered to the subject from about 12 to 72 hours or at any time point in between 12 to 72 hours prior to administration of the reovirus. Thus, the immunosuppressive agent is optionally administered at least 72 hours, at least 48 hours, at least 24 hours or at least 12 hours prior to administration of the reovirus. The reovirus is administered in an effective amount to kill the neoplastic cells of the proliferative disorder. Optionally, the proliferative disorder is a ras-mediated proliferative disorder.

As used herein, the term immunosuppressive agent refers to an agent that inhibits, slows or reverses the activity of the immune system. Immunosuppressive agents act by suppressing the function of responding immune cells (including, for example, T cells), directly (e.g., by acting on the immune cell) or indirectly (by acting on other'mediating cells). Conventional nonspecific immunosuppressive agents include, but are not limited to steroids, glucocorticosteroids, cyclosporine, cyclosporine analogs, cyclophosphamide, corticosteroids including prednisone and methylprednisolone, azathioprine, FK-506 (Fujisawa Pharmaceuticals, Deerfield, Ill.), 15-deoxyspergualin, basiliximab, daclizumab, rapamycin, mycophenolate mofetil, interferons, corticosteroids, sulfasalazine, azathioprine, mimoribine, misoprostol, anti-IL-2 receptor antibodies, thalidomide, anti-tumor necrosis factor antibodies, anti-CD2 antibodies, anti-CD147 antibodies, anti-CD4 antibodies, anti-CD8 antibodies and anti-thymocyte globulin antibodies. Immunosuppressive agents also include ORTHOCLONE® (OKT3) (Ortho Biotech, Raritan, N.J.), SANDIMMUNE® ORAL (cyclosporine) (Sandoz Pharmaceuticals, Hanover, N.J.), PROGRAF® (tacrolimus) (Fujisawa Pharmaceuticals, Deerfield, Ill.), CELLCEPT® (mycophenolate) (Roche Pharmaceuticals, Nutley, N.J.) and RAPAMUNE® (sirolimus) (Wyeth, Collegeville, Pa.). Optionally, the agent is rapamycin, tacrolimus, mycophenolic acid, azathioprine or cyclophosphamide. Optionally, the immunosuppressive agent is not cyclosporin, anti-CD4 antibodies or anti-CD8 antibodies.

As discussed in the examples below, anti-tumor activity was seen in a phase I study of i.v. administered reovirus (REOLYSIN®) to patients with advanced cancer. The patients had a significant increase in neutralizing antibodies after reovirus treatment, except for one patient. These data support the development of protocols in which immune suppressive drugs are combined with systemically administered reovirus in the treatment of cancer. Thus, also provided herein are methods of treating or ameliorating a proliferative disorder in a subject comprising administering to the subject an oncolytic virus and at least one B-cell modulating agent. The B-cell modulating agent is selected from the group consisting of an anti-B-cell antibody, an anti-cytokine antibody and a small molecule selective modulator of B-cells. Optionally, the level of B-cells in the subject is selectively modulated. Optionally, the mammal is immunocompetent. Optionally, the oncolytic virus is immunoprotected. Optionally, a first B-cell modulating agent (e.g., an anti-B-cell antibody) and a second B-cell modulating agent are administered to the subject. The second B-cell modulating agent can be selected from the group consisting of an anti-cytokine antibody and a small molecule selective modulator of B-cells. Optionally, the second B-cell modulating agent is an anti-cytokine antibody.

The level of B-cells in the mammal is modulated prior to contacting the neoplastic cells with the oncolytic virus; concurrent with contacting the neoplastic cells with the oncolytic virus; after contacting the neoplastic cells with the oncolytic virus; and/or while the neoplastic cells are infected with the oncolytic virus. Optionally, the level of B-cells in the subject is reduced by the modulating agent.

As used herein, modulating the level of B-cells in a subject means controlling the level of B-cells, including increasing, decreasing and maintaining the level of B-cells. Selectively modulating the level of B-cells means controlling the level of B-cells to a greater extent than other cells (e.g., other immune cells such as T-cells), typically to a statistically significant extent. The level of selectively modulated B-cells is controlled essentially independently of other immune cells. B-cells refers to B-lymphocytes. There are two major subpopulations of B lymphocytes, B-1 and B-2 cells. B-1 cells are self-renewing and frequently secrete high levels of antibody which binds to a range of antigens (polyspecificity) with a relatively low affinity. The majority of B-cells, B-2 cells, are directly generated from precursors in the bone marrow and secrete highly specific antibody. The modulated level of B-cells can be within ±50% of the normal range of values for B-cells, or within ±25%, ±10%, or ±5% of the normal range of values. Optionally, the modulated level of B-cells can be less than the normal range of values for B-cells. For example, the modulated level of B-cells can be less than 50%, 25%, 10%, 5% or 1% of the normal range of values for B-cells. Optionally, the level of B-cells is reduced such that the neutralizing anti-virus response is reduced, but not ablated, such that the neutralizing anti-virus response is only partially compromised. For example, the neutralizing anti-virus response is reduced 1%, 5%, 10%, 20%, 30%, 40%, 50%, 60%, 70%, 80%, or 90%, or any amount between 1% and 90%.

A B-cell modulating agent (e.g., an anti-B-cell antibody, an anti-cytokine antibody, or a small molecule selective modulator of B-cells) modulates B-cell levels in a mammal and/or can interfere with one or more B-cell functions, e.g. by reducing or preventing a humoral response elicited by the B-cell, by suppressing antibody production, or the like. A selective B-cell modulating agent modulates B-cells to a greater extent than other cells (e.g., other immune cells such as T-cells), typically to a statistically significant extent. Optionally, the modulation of B-cells is controlled essentially independently of other immune cells by the selective agent. The modulating agent preferably is able to deplete B-cells (i.e. reduce circulating B-cell levels) on administration to a subject. Such depletion may be achieved via various mechanisms such antibody-dependent cell mediated cytotoxicity (ADCC) and/or complement dependent cytotoxicity (CDC), inhibition of B-cell proliferation and/or induction of B-cell death (e.g. via apoptosis), or the like. Agents included within the scope of the present disclosure include antibodies, synthetic or native sequence peptides and small molecule antagonists which bind to a B-cell surface antigen, optionally conjugated with or fused to a cytotoxic agent. The preferred antagonist comprises an antibody, more preferably a B-cell depleting antibody.

Optionally, the B-cell modulating agent is an anti-B-cell antibody. The anti-B-cell antibody can be selective for a B-cell including pre-B lymphocytes and mature B lymphocytes. The anti-B-cell antibody can bind to a B-cell antigen, for example, an antigen selected from the group consisting of CD19, CD20, CD22, CD23, CD27, CD37, CD53, CD72, CD73, CD74, CDω78, CD79a, CD79b, CD80, CD81, CD82, CD83, CDw84, CD85 and CD86. Optionally, the anti-B-cell antibody binds to a B-cell antigen selected from the group consisting of CD20 and CD22. Examples of antibodies which bind the CD20 antigen include C2B8 (U.S. Pat. No. 5,736,137); the yttrium-[90]-labeled 2138 murine antibody designated Y2B8 (U.S. Pat. No. 5,736,137); murine IgG2a 131 optionally labeled with 1311 to generate the 1311-B1 antibody (U.S. Pat. No. 5,595,721); murine monoclonal antibody 1F5 (Press et al. Blood 69(2):584-591 (1987)); chimeric 2H7 antibody (U.S. Pat. No. 5,677,180); and monoclonal antibodies L27, G28-2, 93-1133, B-C1 or NU-B2 available from the International Leukocyte Typing Workshop (Valentine et al., In: Leukocyte Typing III (McMichael, Ed., p. 440, Oxford University Press (1987)). Examples of antibodies which bind the CD19 antigen include the anti-CD19 antibodies in Hekman et al., Cancer Immunol. Immunother. 32:364-372 (1991) and Vlasveld et al. Cancer Immunol. Immunother. 40:37-47 (1995); and the B4 antibody in Kiesel et al. Leukemia Research 11, 12: 1119 (1987).

Optionally, the B-cell modulating agent is an anti-cytokine antibody. An anti-cytokine antibody is an antibody that modulates the expression and/or activity of a cytokine. The term cytokine includes proteins released by one cell population which act on another cell as intercellular mediators. As used herein, the term cytokine includes proteins from natural sources or from recombinant cell culture and biologically active equivalents of the native sequence cytokines. Examples of cytokines are lymphokines, monokines, interleukins, interferons and traditional polypeptide hormones. Typical cytokines include interleukins (ILs) such as IL-1, IL-1a, IL-2, IL-3, IL-4, IL-5, IL-6, IL-7, IL-8, IL-9, IL-11, IL-12, IL-15; interferon alpha and interferon gamma (IFN-alpha and IFN-gamma); or a tumor necrosis factor such as TNF-a or TNF-β. Thus, the anti-cytokine antibody can bind to a cytokine selected from the group consisting of IL2, IL6, IL10, IFN-gamma and TNF-alpha. Optionally, the anti-cytokine antibody binds to IL10.

Optionally, the B-cell modulating agent is a small molecule selective modulator of B-cells. A small molecule selective modulator of B-cells includes small organic molecules and peptides which modulate B-cells. Examples include aromatic quinolinecarboxamides (e.g., quinoline-8-arylcarboxamides such as N-[4-(Trifluoromethyl)phenyl]quinoline-8-carboxamide, N-[5-(Trifluoromethyl)pyridin-2-yl]quinole-8-carboxamide, N-(Phenyl)quinoline-8-carboxamide, N-[2-(Trifluoromethyl)phenyl]quinoline-8-carboxamide, N-[3-(Trifluoromethyl)phenyl]quinoline-8-carboxamide, 7-Methyl-N-[4-(trifluoromethyl)phenyl]quinoline-8-carboxamide, N-[4-(Trifluoromethyl)phenyl]-2-(trifluoromethyl)quinoline-8-carboxamide, or N-[4-(Trifluoromethyl)phenyl]-1,2,3,4-tetrahydroquinoline-8-carboxamide; see Papageorgiou et al., J Med. Chem. 2001 Jun. 7; 44(12):1986-92); dexamethasone (Cupic et al, J. Int J Mol. Med. 2005 June; 15(6):1023-31); rapamycin, leflunomide, mizoribine, mycophenolic acid, brequinar, and deoxyspergualin (Morris, J Heart Lung Transplant. 1993 November-December; 12(6 Pt 2):S275-86); FK506, rapamycin, mycophenolate mofetil malononitrilamides (leflunomide, FK778, FK779), 15-deoxyspergualin (DSG) (Ma et al., Curr Drug Targets Cardiovasc Haematol Disord. 2002 December; 2(2):57-71); sulfasalazine and its analogs (Habens et al., Apoptosis. 2005 May; 10(3):481-91); genistein (Mansour et al., Cell Cycle. 2004 December; 3(12):1597-605); 2-cyano-3,12-dioxooleana-1,9-dien-28-oic acid (Ray et al., Exp Hematol. 2006 September; 34(9):1201-1210); PD 0332991 (Baughn et al., Cancer Res. 2006 Aug. 1; 66(15):7661-7); BU12-SAPORIN (Flavell et al., Br J Haematol. 2006 July; 134(2):157-70. Epub 2006 Jun. 12); HA14-1 (Skommer et al., Leuk Res. 2006 March; 30(3):322-31); PKC412 (Chen et al., Oncogene. 2005 Dec. 15; 24(56):8259-67); PS-1145 and related compounds (Lam et al., Clin Cancer Res. 2005 Jan. 1; 11(1):28-40); (−)-Gossypol (Mohammad et al., Mol Cancer Ther. 2005 January; 4(1):13-21); 4-amino-1-tert-butyl-3-(1-naphthyl)pyrazolo[3,4-d]pyrimidine (Wong et al., Proc Natl Acad Sci USA. 2004 Dec. 14; 101(50):17456-61); dipeptidyl peptidase (DPP) inhibitors, e.g., PT-100 (Val-boro-Pro) (Adams et al., Cancer Res. 2004 Aug. 1; 64(15):5471-80); and the like.

Optionally, the small molecule selective modulator of B-cells is selected from the group consisting of aromatic quinoline carboxamides, dexamethasone, leflunomide, mizoribine, brequinar, deoxyspergualin, malononitrilamides, sulfasalazine, genistein, 2-cyano-3,12-dioxooleana-1,9-dien-28-oic acid, PD 0332991, BU12-SAPORIN, HA14-1, PKC412, PS-1145, gossypol, 4-amino-1-tert-butyl-3-(1-naphthyl)pyrazolo[3,4-d]pyrimidine, and Val-boro-Pro.

Optionally, the B-cell modulating agent and the small molecule selective modulator of B-cells specifically exclude one or more compounds selected from the group consisting of rapamycin, tacrolimus, and mycophenolic acid, and analogs thereof. Optionally, the B-cell modulating agent and the small molecule selective modulator of B-cells specifically exclude one or more compounds selected from the group consisting of 5-fluorouracil, mitomycin C, methotrexate, hydroxyurea, cyclophosphamide, dacarbazine, mitoxantrone, anthracyclins, carboplatin, cisplatin, taxol, taxotere, tamoxifen, anti-estrogens, interferons, rapamycin, tacrolimus, mycophenolic acid, azathioprine, cyclosporin, cyclophosphamide and analogs thereof.

An oncolytic virus, as used herein, is a virus which is capable of selectively replicating in neoplastic cells. Optionally, the oncolytic virus is selected from the group consisting of reovirus, modified adenovirus, modified HSV, modified vaccinia virus, modified parapoxvirus orf virus, modified influenza virus, delNS1 virus, p53-expressing viruses, ONYX-015, Delta24, and vesicular stomatitis virus.

The term modified adenovirus refers to an adenovirus in which the gene product or products which prevents the activation of PKR is lacking, inhibited or mutated such that PKR activation is not blocked. Preferably, the VAI RNA's are not transcribed. Such modified adenovirus would not be able to replicate in normal cells that do not have an activated ras pathway; however, it would be able to infect and replicate in cells having an activated ras pathway.

The term modified HSV refers to a herpes simplex virus (HSV) in which the gene product or products that prevents the activation of PKR is lacking, inhibited or mutated such that PKR activation is not blocked. Preferably, the HSV gene γ134.5 is not transcribed. Such modified HSV would not be able to replicate in normal cells that do not have an activated ras pathway; however, it would be able to infect and replicate in cells having an activated ras pathway.

Parapoxvirus orf virus is a poxvirus. It is a virus that induces acute cutaneous lesions in different mammalian species, including humans. Parapoxvirus orf virus naturally infects sheep, goats and humans through broken or damaged skin, replicates in regenerating epidermal cells and induces pustular lesions that turn to scabs. The term modified parapoxvirus orf virus refers to a parapoxvirus orf virus in which the gene product or products which prevents the activation of PKR is lacking, inhibited or mutated such that PKR activation is not blocked. Preferably, the gene OV20.0L is not transcribed. Such modified parapoxvirus orf virus would not be able to replicate in normal cells that do not have an activated ras pathway; however, it would be able to infect and replicate in cells having an activated ras pathway.

The term modified vaccinia virus refers to a vaccinia virus in which the gene product or products which prevents the activation of PKR is lacking, inhibited or mutated such that PKR activation is not blocked. Preferably, the E3L gene and/or the K3L gene is not transcribed. Such modified vaccinia virus would not be able to replicate in normal cells that do not have an activated ras pathway; however, it would be able to infect and replicate in cells having an activated ras pathway.

Optionally, the oncolytic virus can be a recombinant virus, e.g., a recombinant reovirus. The neoplastic cells can be contacted with more than one type of oncolytic virus, or more than one strain of oncolytic virus. The oncolytic virus can also be encapsulated in a micelle. The oncolytic virus can be treated with a protease prior to contacting the neoplastic cells.

Optionally, the method further includes administering anti-anti-oncolytic virus antibodies to the mammal; or removing anti-oncolytic virus antibodies from the mammal. An anti-oncolytic virus antibody refers to an antibody which binds to oncolytic virus. IgG antibodies refers to immunoglobulin G antibodies. IgG, the most abundant type of antibody, carries the major burden of neutralizing bacterial toxins and binding to microorganisms to enhance their phagocytosis. Selective removal of anti-oncolytic virus antibodies can prevent the subject's immune system from removing therapeutically administered oncolytic virus. Preventing antibody interaction with the virus may also assist systemic treatment strategies. Antibodies can be removed by several methods, including heme-dialysis and passing the blood over immobilized oncolytic virus (selective antibody removal); by removal of all IgG antibodies by heme-dialysis and passing the blood over immobilized protein A (commercially available as PROSORBA, Cypress Bioscience, San Diego, Calif.); or by administration of humanized anti-idiotypic antibodies, where the idiotype is against the oncolytic virus.

Optionally, the oncolytic virus can act systemically without impairing normal immune function by masking or impairing immune recognition of the oncolytic virus. To prevent the subject's immune system from recognizing the oncolytic virus, the serial administration of the oncolytic virus and oncolytic virus reassortants is performed. Alternatively, the oncolytic virus may be coated with non-virotoxic humanized antibodies, such as coating with the Fab portion of the antibody, or coated in a micelle.

Additionally, the oncolytic virus may be treated with chymotrypsin to yield an infectious subviral particle (ISVP). An ISVP may be used either alone or in combination with whole virus to provide an agent that is either poorly recognized has not been previously prevented by the patient's immune system.

The oncolytic virus contains different antigenic determinants thereby reducing or preventing an immune response by a mammal previously exposed to an oncolytic virus subtype. Such recombinant virions, also known as reassortants, can be generated by co-infection of mammalian cells with different subtypes of oncolytic virus with the resulting resorting and incorporation of different subtype coat proteins into the resulting virion capsids.

The oncolytic virus is preferably an oncolytic virus modified to reduce or eliminate an immune reaction to the oncolytic virus. Such modified oncolytic viruses are termed immunoprotected oncolytic viruses. Such modifications could include packaging of the oncolytic virus in a liposome, a micelle or other vehicle to mask the oncolytic virus from the mammals immune system. Alternatively, the outer capsid of the oncolytic virus virion particle may be removed since the proteins present in the outer capsid are the major determinant of the host humoral and cellular responses.

Also provided herein are pharmaceutical compositions and kits comprising an oncolytic virus and at least one B-cell modulating agent. The B-cell modulating agent is selected from the group consisting of an anti-B-cell antibody, an anti-cytokine antibody and a small molecule selective modulator of B-cells. The pharmaceutical composition or kit optionally includes both a first B-cell modulating agent (e.g., an anti-B-cell antibody) and a second B-cell modulating agent. The pharmaceutical composition or kit also optionally includes a chemotherapeutic agent or a non-B-cell specific immune suppressive agent. Optionally, the non-B-cell specific immune suppressive agent can be a steroid, or can be selected from the group consisting of rapamycin, tacrolimus, mycophenolic acid, azathioprine, cyclosporin, cyclophosphamide and analogs thereof. Preferably, the non-B-cell specific immune suppressive agent is cyclosporin.

In the provided methods, the term proliferative disorder refers to a disorder characterized by neoplastic or tumor cells. A neoplastic cell, tumor cell, or cell with a proliferative disorder refers to a cell which proliferates at an abnormally high rate. A new growth comprising neoplastic cells is a neoplasm, also known as a tumor. A tumor is an abnormal tissue growth, generally forming a distinct mass, that grows by cellular proliferation more rapidly than normal tissue growth. A tumor may show a partial or total lack of structural organization and functional coordination with normal tissue. As used herein, a tumor is intended to encompass hematopoietic tumors as well as solid tumors. As used herein, the term proliferative disorder includes tumors, neoplasms, and disorders characterized by neoplastic or tumor cells or cells which proliferate at an abnormally high rate.

A tumor may be benign (benign tumor) or malignant (malignant tumor or cancer). Malignant tumors are broadly classified into three major types. Malignant tumors arising from epithelial structures are called carcinomas; malignant tumors that originate from connective tissues such as muscle, cartilage, fat, or bone are called sarcomas; and malignant tumors affecting hematopoietic structures (structures pertaining to the formation of blood cells) including components of the immune system are called leukemias and lymphomas. Other tumors include, but are not limited to, neurofibromatosis.

Optionally, the neoplastic cells are ras-mediated. The terms ras-activated and ras-mediated are used throughout interchangeably. The ras pathway may be activated by way of ras gene mutation, elevated level of ras gene expression, elevated stability of the ras gene message, or any mutation or other mechanism which leads to the activation of ras or a factor or factors downstream or upstream from ras in the ras pathway, thereby increasing the ras pathway activity. For example, activation of an EGF receptor, PDGF receptor or SOS results in activation of the ras pathway. Ras-mediated neoplastic cells include, but are not limited to, ras-mediated cancer cells, which are cells proliferating in a malignant manner due to activation of the ras pathway. Optionally, in the ras-mediated neoplastic cells, the phosphorylation of PKR is prevented. For example, the phosphorylation of PKR can be prevented by inactivation or deletion of PKR. The ras-mediated neoplastic cells can be devoid of PKR or phosphorylation of PKR in the ras-mediated neoplastic cells can be prevented or reversed. Ras-activated neoplastic cells or ras-mediated neoplastic cells refer to cells which proliferate at an abnormally high rate due to, at least in part, activation of the ras pathway.

As used herein the term ras-mediated proliferative disorder refers to a disorder characterized by ras-activated or ras-mediated neoplastic cells. Thus, for example, a ras-mediated proliferative disorder includes a neoplasm wherein the neoplasm contains ras-mediated proliferative cells.

The proliferative disorder is optionally a neoplasm or a solid neoplasm. Neoplasms include solid tumors, for example, carcinomas and sarcomas. Carcinomas include malignant neoplasms derived from epithelial cells which infiltrate or invade surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue. Provided herein are methods of treatment of proliferative disorders of myeloid or lymphoid systems, including leukemias, such as acute lymphocytic leukemia, acute nonlymphocytic leukemia, chronic lymphocytic leukemia, chronic myelocytic leukemia, and hairy cell leukemia, lymphomas, including effusion lymphomas and body cavity based lymphomas and other cancers that typically are not present as a tumor mass but are distributed in the vascular or lymphoreticular systems.

The proliferative disorder includes an adult or pediatric proliferative disorders; a growth of solid tumors/malignancies; myxoid and round cell carcinoma; locally advanced tumors; thyroid cancer; adrenal cancer; liver cancer; pancreatic cancer; central and peripheral nervous system cancer; human soft tissue sarcomas, including Ewing's sarcoma; cancer metastases, including lymphatic metastases; squamous cell carcinoma, particularly of the head and neck; esophageal squamous cell carcinoma; oral carcinoma; blood cell malignancies including multiple myeloma; lung cancer, including small cell carcinoma of the lungs; cutaneous T cell lymphoma; Hodgkin's lymphoma; non-Hodgkin's lymphoma; cancer of the adrenal cortex; ACTH-producing tumors; non-small cell lung cancers; breast cancer; gastro-intestinal cancers, including stomach cancer, colon cancer, colorectal cancer, and polyps associated with colorectal neoplasia; pancreatic cancer; liver cancer; urological cancers, including bladder cancer, such as primary superficial bladder tumors, invasive transitional cell carcinoma of the bladder, and muscle-invasive bladder cancer; prostate cancer; malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle; malignancies of the male genital tract, including testicular cancer and penile cancer; kidney cancer, including renal cell carcinoma; brain cancer, including intrinsic brain tumors, neuroblastoma, astrocytic brain tumors, gliomas, and metastatic tumor cell invasion in the central nervous system; bone cancers, including osteomas and osteosarcomas; skin cancers, including malignant melanoma, tumor progression of human skin keratinocytes, basal cell carcinoma, and squamous cell cancer; thyroid cancer; retinoblastoma; neuroblastoma; peritoneal effusion; malignant pleural effusion; mesothelioma; Wilms's tumors; gall bladder cancer; trophoblastic neoplasms; hemangiopericytoma; and Kaposi's sarcoma.

The herein provided viruses and agents are administered in vitro or in vivo, optionally, in a pharmaceutically acceptable carrier or excipient. By pharmaceutically acceptable is meant a material that is not biologically or otherwise undesirable, i.e., the material are administered to a subject, without causing undesirable biological effects or interacting in a deleterious manner with other components of the pharmaceutical composition in which it is contained. The carrier is selected to minimize any degradation of the active ingredient and to minimize any adverse side effects in the subject.

Thus, provided are pharmaceutical compositions which contain viruses and/or agents. In making the pharmaceutical compositions, the viruses and/or agents are usually mixed with an excipient, diluted by an excipient or enclosed within such a carrier which can be in the form of a capsule, sachet, paper or other container. When the pharmaceutically acceptable excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. Suitable formulations can be found in Remington: The Science and Practice of Pharmacy (21st ed.) eds. A. R. Gennaro et al., University of the Sciences in Philadelphia 2005.

Some examples of suitable excipients include lactose, dextrose, sucrose. sorbitol, mannitol, starches, gum acacia, calcium phosphate, alginates, tragacanth. gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the subject by employing procedures known in the art.

The virus can be encapsulated in a liposome, micelle, microparticle, nanoparticle, or microsphere using methods and materials known to those of skill in the art. For example, U.S. Pat. No. 5,019,400 describes the preparation of controlled release biodegradable microspheres made of various materials. Materials for liposomes include lipids, block co-polymers, and other biologically compatible surfactants and copolymers thereof. Methods for making microparticles include, for example, dissolving, emulsifying or suspending a polymer liquid that contains the molecule of interest.

For preparing solid compositions such as tablets, the active ingredient(s) is/are mixed with a pharmaceutical excipient to form a solid preformulation composition containing a homogeneous mixture of the active ingredient(s). When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills are optionally coated or otherwise compounded to provide a dosage form affording the advantage of prolonged action. For example, the tablet or pill comprises an inner dosage and an outer dosage component, the latter being in the form of an envelope over the former. The two components are optionally separated by an enteric layer which serves to resist disintegration in the stomach and permit the inner component to pass intact into the duodenum or to be delayed in release. A variety of materials are used for such enteric layers or coatings, such materials including a number of polymeric acids and mixtures of polymeric acids with such materials as shellac, cetyl alcohol, and cellulose acetate.

The liquid forms in which the compositions are incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid composition's optionally contain suitable pharmaceutically acceptable excipients as described herein. Preferably the compositions are administered by the oral or nasal respiratory route for local or systemic effect. Compositions in preferably pharmaceutically acceptable solvents are optionally nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

Another formulation used in the methods provided herein employs transdermal delivery devices (patches). The construction and use of transdermal patches for the delivery of pharmaceutical agents is well known in the art. See, for example, U.S. Pat. No. 5,023,252, herein incorporated by reference. Such patches are constructed for continuous, pulsatile, or on demand delivery of pharmaceutical agents.

In the provided methods, the virus is administered in a manner so that it can ultimately contact the target tumor or tumor cells. For example, the virus is administered systemically. The route by which the virus is administered, as well as the formulation, carrier or vehicle, depend on the location as well as the type of the target cells. A wide variety of administration routes are employed. For example, for a solid tumor that is accessible, the virus is optionally administered by injection directly to the tumor. For a hematopoietic tumor, for example, the virus is administered intravenously or intravascularly. For tumors that are not easily accessible within the body, such as metastases, the virus is administered in a manner such that it is transported systemically through the body of the mammal and thereby reaching the tumor (e.g., intravenously or intramuscularly). Alternatively, the virus is administered directly to a single solid tumor, where it then is carried systemically through the body to metastases. The virus is optionally administered subcutaneously, intraperitoneally, intrathecally (e.g., for brain tumor), topically (e.g., for melanoma), orally (e.g., for oral or esophageal cancer), rectally (e.g., for colorectal cancer), vaginally (e.g., for cervical or vaginal cancer), nasally or by inhalation spray (e.g., for lung cancer).

In the provided methods, the agent or agents and compositions are administered orally, parenterally (e.g., intravenously), by intramuscular injection, by intraperitoneal injection, transdermally, extracorporeally, topically or the like, including intranasal administration or administration by inhalant.

The virus is administered in an amount that is sufficient to treat the tumor or neoplasm (e.g., an effective amount). Treatment results, for example, in a reduction in size of the neoplasm, or in a complete elimination of the neoplasm. The reduction in size of the neoplasm, or elimination of the neoplasm, is generally caused by lysis of neoplastic cells (oncolysis) by the oncolytic virus. The effective amount is from about 1.0 to about 10¹⁵ pfu/kg body weight, or any amount in between 1.0 and 10¹⁵ pfu/kg body weight. Optionally, the effective amount of reovirus is from about 10³ to about 10¹² pfu/kg body weight or about 10⁸ to 10¹² pfu/kg body weight. Thus, approximately 1.0 to 10¹⁵ plaque forming units (PFU) of virus can be used, depending on the type, size and number of tumors present.

The virus is optionally formulated in a unit dosage form, each dosage containing from about 10² pfus to about 10¹⁷ pfus of the reovirus. The term unit dosage forms refers to physically discrete units suitable as unitary dosages for subjects, each unit containing a predetermined quantity of oncolytic virus calculated to produce the desired therapeutic effect, in association with a suitable pharmaceutical excipient.

Effective dosages of compositions depends on a variety of factors and may thus vary somewhat from subject to subject. The exact amount required varies from subject to subject, depending on the species, age, weight and general condition of the subject, the severity of the disease being treated, the particular virus or vector used and its mode of administration. Thus, it is not possible to specify an exact amount for every composition. However, an appropriate amount is determined by one of ordinary skill in the art using only routine experimentation given the guidance provided herein.

The dosage ranges for the administration of the compositions are those large enough to produce the desired effect in which the symptoms of the disease are affected. The dosage is not so large as to cause adverse side effects, such as unwanted cross-reactions and anaphylactic reactions. The dosage is adjusted by the individual physician in the event of any counter indications.

The virus is administered in a single dose or multiple doses (i.e. more than one dose) in one dosing schedule. For multiple doses, the dosing schedule can occur over a period of days or weeks. The dosing schedule is optionally repeated weekly or monthly, as appropriate. The reovirus is optionally administered to more than one neoplasm in the same subject simultaneously or in series.

The provided methods are optionally combined with other tumor therapies such as chemotherapy, radiotherapy, surgery, and/or hormone therapy. Chemotherapeutic agents are compounds which may inhibit the growth of tumors. Such agents, include, but are not limited to. 5-fluorouracil, mitomycin C, methotrexate, hydroxyurea, cyclophosphamide, dacarbazine, mitoxantrone, anthracyclins (Epirubicin and Doxurubicin), antibodies to receptors, such as herceptin, etopside, pregnasome, platinum compounds such as carboplatin and cisplatin, taxanes such as taxol and taxotere, hormone therapies such as tamoxifen and anti-estrogens, interferons, aromatase inhibitors, progestational agents and LHRH analogs. Thus, the provided compositions are optionally administered in combination with one or more other therapeutic or prophylactic regimens.

Provided herein are kits for treating proliferative disorders in a subject. For example, the kits comprise one or more reoviruses and one or more immunosuppressive agents. Alternatively, the kits comprise a composition comprising a one or more reoviruses and a composition comprising one or more immunosuppressive agents. Optionally, the reovirus, the immunosuppressive agent or compositions of the reovirus and/or immunosuppressive agent are provided in the kit in dosage units. For example, one dosage unit of immunosuppressive agent and up to 5 dosage units of reovirus are provided in a kit.

Also provided herein are kits comprising one or more oncolytic viruses and one or more B-cell modulating agents. Alternatively, the kits comprise a composition comprising a one or more oncolytic viruses and a composition comprising one or more B-cell modulating agents. Optionally, the compositions are provided in the kit in dosage units.

The provided kits optionally comprise, for example, another pharmaceutically active agent, such as, for example, a chemotherapeutic agent, a buffering agent, preservative, protein-stabilizing agent or additional agents or components necessary for carrying out the provided methods. Each component of the kit is usually enclosed within an individual container. However, two or more components of the kit can be contained within one container. In addition, all of the various containers can be contained within one or more packages. Kits optionally include instructions for use of the components of the kit.

As used herein the terms treatment, treat, treating or ameliorating refers to a method of reducing the effects of a disease or condition or one or more symptoms of the disease or condition. These terms also refers to slowing the rate of progression of one or more symptoms of the disease or condition. Thus in the disclosed method treatment can refer to a 10%, 20%, 30%, 40%, 50%, 60%, 70%, 80%, 90% or 100% reduction or amelioration in the severity of an established disease or condition or symptom of the disease or condition. For example, the method for treating proliferative disorders is considered to be a treatment if there is a 10% reduction in one or more symptoms or 10% reduction in the rate of progression of one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the term subject includes a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. As used herein, patient or subject may be used interchangeably and can refer to a subject afflicted with a disease or disorder. The term patient or subject includes human and veterinary subjects.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

### Examples

Anti-tumor activity was seen in a phase I study of i.v. administered reovirus (REOLYSIN®) to patients with advanced cancer. Patients were treated with 1×10⁸, 3×10⁸, 1×10⁹, 3×10⁹, 1×10¹⁰, or 3×10¹⁰ (TCID50) of reovirus. The patients had a significant increase in neutralizing antibodies after reovirus treatment, with peak endpoint titres >1/10000, except for one patient. The median fold increase was 250, with a range of 9-6437. Serum from patients in four different dose cohorts was used to neutralize reovirus cytotoxicity effect against L929 cells. Serum from the patient yet to be treated with reovirus was as effective as goat polyclonal antibody in neutralizing reovirus cytotoxicity against L929 cells suggesting the presence of preexisting immunity. However, some patient serum did not show immunity to reovirus.

The above results support the development of protocols in which immune suppressive drugs are combined with systemically administered reovirus in the treatment of cancer.

1. A method of treating a proliferative disorder in a subject, the method comprising the steps of:
a) administering to the subject one dose of an immunosuppressive agent; and
b) administering to the subject one to five doses of a reovirus, wherein the immunosuppressive agent is administered at least about 72 hours prior to administration of the reovirus.

2. The method of claim 1, wherein the immunosuppressive agent is administered from about 96 to 72 hours prior to administration of the reovirus.

3. The method of claim 1, wherein the immunosuppressive agent is administered at 72 or at 96 hours prior to administration of the reovirus.

4. (canceled)

5. The method of claim 1, wherein the immunosuppressive agent is not cyclosporine A, an anti-CD4 antibody or an anti-CD8 antibody.

6. The method of claim 1, wherein the immunosuppressive agent is selected from the group consisting of rapamycin, tacrolimus, mycophenolic acid, azathioprine, cyclophosphamide and analogs thereof.

7. (canceled)

8. The method of claim 1, wherein one, two, three, four or five doses of reovirus is are administered.

9. (canceled)

10. (canceled)

11. (canceled)

12. (canceled)

13. The method of claim 1, wherein the reovirus is administered systemically.

14. The method of claim 1, wherein the reovirus is administered orally, topically, nasally, subcutaneously or intraperitoneally.

15. The method of claim 1, wherein the dose of reovirus is about 1 to 10¹⁵ plaque forming units of reovirus/kg body weight.

16. The method of claim 15, wherein the dose of reovirus is about 10⁸ to 10¹² plaque forming units of reovirus/kg body weight.

17. The method of claim 1, wherein the reovirus is administered by injection into or near the site of the proliferative disorder.

18. The method of claim 1, wherein the proliferative disorder is a neoplasm.

19. The method of claim 18, wherein the neoplasm is a solid neoplasm.

20. (canceled)

21. The method of claim 1, wherein the reovirus is administered in an effective amount to kill the neoplastic cells of the proliferative disorder.

22. The method of claim 1, wherein the proliferative disorder is a ras-mediated proliferative disorder.

23. The method of claim 1, wherein the reovirus is a mammalian reovirus.

24. The method of claim 1, wherein the reovirus is a human reovirus.

25. The method of claim 1, wherein the reovirus is selected from the group consisting of serotype 1 reovirus, serotype 2 reovirus and serotype 3 reovirus.

26. (canceled)

27. The method of claim 1, wherein the reovirus is a recombinant reovirus.

28. (canceled)

29. The method of claim 1, wherein the reovirus is an immunoprotected reovirus or a modified reovirus.

30. (canceled)

31. The method of claim 1, wherein the reovirus is a chemically modified reovirus, comprises mutated coat proteins or is encapsulated in a liposome or micelle.

32. The method of claim 1, wherein the reovirus is chemically or biochemically treated prior to administration to the subject.

33. (canceled)

34. (canceled)

35. The method of claim 1, wherein the reovirus is a reassortant reovirus.

36. The method of claim 1, wherein the reovirus has enhanced oncolytic activity.

37. The method of claim 1, wherein more that one type or more than one strain of reovirus is administered.

38. (canceled)

39. The method of claim 1, wherein the reovirus has IDAC Accession No. 190907-01.

40. The method of claim 1, wherein the reovirus comprises a lambda-3 polypeptide having one or more amino acid modifications.

41. The method of claim 1, wherein the reovirus comprises a L1 genome segment comprising one or more nucleic acid modifications.

42. A method of treating a proliferative disorder in a subject, the method comprising the steps of:
a) administering to the subject an oncolytic virus; and
b) administering to the subject at least one B-cell modulating, wherein the B-cell modulating agent selected from the group consisting of an anti-B-cell antibody, an anti-cytokine antibody and a small molecule selective modulator of B-cells.

43. A kit comprising an oncolytic virus and a B-cell modulating agent, wherein the B-cell modulating agent is selected from the group consisting of an anti-B-cell antibody, an anti-cytokine antibody and a small molecule selective modulator of B-cells.
